(19) United States
(12) Patent Application Publication
(10) Pub. No.: US 2025/0388734 A1
(43) Pub. Date: Dec. 25, 2025

(54) A PROCESS FOR RECYCLING ONE OR MORE POLYMERS, SUCH AS POLYURETHANES, CONTAINED IN A SOLID MATERIAL

(71) Applicant: BASF SE, Ludwigshafen am Rhein (DE)

(72) Inventors: Patrick SCHMATZ-ENGERT, Ludwigshafen am Rhein (DE); Jens FERBITZ, Ludwigshafen am Rhein (DE); Matthias Maximilian NIEDERMAIER, Ludwigshafen am Rhein (DE); Torsten MATTKE, Ludwigshafen am Rhein (DE); Christian DITTRICH, Ludwigshafen am Rhein (DE); Michaela STUPPY, Ludwigshafen am Rhein (DE)

(21) Appl. No.: 18/880,686

(22) PCT Filed: Jul. 4, 2023
(86) PCT No.: PCT/EP2023/068412

§ 371 Date: Jan. 2, 2025

(30) Foreign Application Priority Data
Jul. 5, 2022 (EP) 22183137.3

(51) Int. Cl. C08J 11/28; C08J 11/16

(52) U.S. Cl. CPC C08J 11/28; C08J 11/16; C08J 2375/04

(57) ABSTRACT

The present invention relates to a process for recycling one or more polymers selected from the group consisting of polyurethanes, polyurethane ureas, polyisocyanurates, and a mixture of two or more thereof, said one or more polymers being contained in a solid material W.

The present invention relates to a process for recycling one or more polymers selected from the group consisting of polyurethanes, polyurethane ureas, polyisocyanurates, and a mixture of two or more thereof, said one or more polymers being contained in a solid material W.

Polyurethanes belong to the class of polycondensation polymers. They are generally produced from one or more polyhydroxyl compounds and one or more di-or polyisocyanates.

In terms of sustainability, the polyurethanes should be recycled as much as possible. Generally, polyurethanes can be recycled in a variety of ways. The re-monomerization of polyurethane or polyurethane waste with the recovery of polyol and isocyanate components or corresponding precursors is one of the most interesting ways. For this purpose, the prior art proposes the cleavage of the polyurethanes by, for example, hydrolysis, glycolysis, alcoholysis and aminolysis, to form an isocyanate derivative (amine, carbamate, urea) and the polyol.

For example, WO 2008/014988 A1 relates to the redissociation of polyurethanes. In particular, a process is described for splitting polyurethanes and polyurethaneureas, in which the polymer is first reacted with gaseous or liquid secondary aliphatic or cycloaliphatic amines, the secondary urea formed, after removal, is split with hydrogen chloride to the isocyanate. Further, the polyols or polyamines also formed in the reaction are worked up and purified.

One of the biggest challenges of the prior art recycling processes is the separation of the polyols from the isocyanate derivatives, which is mostly based on a phase decay. However, this can be applied only for selected polyurethanes and it can easily fail due to possible contamination in the polyurethane waste. Thus, the prior art recycling processes are typically severely limited with respect to their applicability.

Thus, there is a need for an improved process for recycling polymers such as polyurethanes, polyurethane ureas and polyisocyanurates. Accordingly, it was an object of the present invention to provide an improved process for the recycling of polymers such as polyurethanes, polyurethane ureas and polyisocyanurates, in particular for the decomposition of said polymers into monomers in order to produce new polymers.

Surprisingly, it has been found that the process of the present invention permits to recycle a variety of polyurethanes, polyurethane ureas, polyisocyanurates from solid waste material, such as end-of-life foam, by decomposing said polymers which can then be used for different applications such as the production of new monomers. Thus, the process of the present invention permits to reduce the CO₂ footprint as new polymers can be formed from waste.

Therefore, the present invention relates to a process for recycling one or more polymers selected from the group consisting of polyurethanes, polyurethane ureas, polyisocyanurates, and a mixture of two or more thereof, said one or more polymers being contained in a solid material

W, the process comprising
(i) providing the solid material W containing the one or more polymers;
(ii) introducing the solid material W and one or more primary amines in a reactor unit R_{A}, obtaining a mixture;
(iii) subjecting the mixture obtained according to (ii) in R_{A} to aminolysis reaction conditions, obtaining a mixture M comprising one or more polyurea-containing compounds, and further comprising one or more polyols;
(iv) isolating the one or more polyurea-containing compounds of M obtained according to (iii) from the one or more polyols of M obtained according to (iii), obtaining a mixture U comprising the one or more polyurea-containing compounds and a mixture P comprising the one or more polyols;
(v) subjecting the mixture U comprising the one or more polyurea-containing compounds obtained according to (iv) to cleavage reaction conditions in a reactor unit R_{c}, obtaining either one or more corresponding polyamines or one or more corresponding polyisocyanates.

Preferably, the process further comprises, prior to (i), subjecting the solid material W to a pretreatment, more preferably a mechanical pre-treatment, wherein the mechanical pre-treatment preferably comprises one or more of milling, crushing, shredding and cutting, more preferably milling or shredding, the solid material W.

Preferably, the process further comprises, prior to (i), more preferably after subjecting the solid material W to a pre-treatment as defined in the foregoing, drying the solid material W, wherein drying is more preferably conducted at a temperature in the range of from 40 to 100° C., more preferably in the range of from 50 to 85° C., and wherein more preferably drying is conducted in a gas atmosphere comprising one or more of nitrogen and oxygen, more preferably air.

Preferably the water content of the solid material W, more preferably after drying as defined herein above, is lower than 1000 ppm-weight-%, lower than 100 ppm-weight-%.

Preferably, the solid material W is a waste solid material.

Preferably the waste material is one or more of an end-of-life material, such as an end-of-life foam, end-of-life flexible foam, end-of-life rigid foam, an end-of-life compact elastomer, and end-of-life compact duromer. In the context of the present invention, an end-of-life material is a material at the end of the product lifecycle.

Preferably the solid material W comprises, in addition to the one or more polymers, impurities which can be one or more of glass, sand, wood, metals, papers, inorganic solids and polymers other than polyurethanes, polyurethane ureas and polyisocyanurates. The polymers other than polyurethane, polyurethane urea and polyisocyanurate can be for example one or more of polyethylene (PE), polypropylene (PP), polyethylene terephthalate (PET) or polystyrene (PS). Preferably, the solid material W is one or more of a powder, pieces of foam, pellets, granulates and flakes.

Preferably the average particle size of the solid material W provided in (i) is in the range of from 10⁻⁶ to 10⁻¹ m, preferably in the range of from 10⁻⁶ to 10⁻² m, the average particle size being determined by laser diffraction or light microscopy. These methods being adapted by the skilled person depending on the particle size range. Thus, particle sieve analysis could also be used for determining the average particle size.

Preferably, the one or more polymers contained in the solid material W are one or more polyurethanes, more preferably the one or more polyurethanes are thermosets or elastomers.

According to the present invention, there is no particular restriction as to the polyurethanes of W which are used in the process of the present invention. However, preferably, the polyurethanes of W are prepared by a process wherein polyisocyanates are reacted with polyols in the presence of catalyst(s) as well-known in the art.

Suitable polyisocyanate components used for the production of the polyurethanes of W comprise any of the polyisocyanates known for the production of polyurethanes. These comprise the aliphatic, cycloaliphatic, and aromatic difunctional or polyfunctional isocyanates known from the prior art, and also any desired mixtures thereof. Examples are diphenylmethane 2, 2′-, 2,4′-, and 4,4′-diisocyanate, the mixtures of monomeric diphenylmethane diisocyanates with diphenylmethane diisocyanate homologs having a larger number of rings (polymer MDI), isophorone diisocyanate (IPDI) and its oligomers, tolylene 2,4- and 2,6-diisocyanate (TDI), and mixtures of these, tetramethylene diisocyanate and its oligomers, hexamethylene diisocyanate (HDI) and its oligomers, naphthylene diisocyanate (NDI), and mixtures thereof.

Preferably, tolylene 2,4- and/or 2,6-diisocynate (TDI) or a mixture thereof, monomeric diphenylmethane diisocyanates, and/or diphenylmethane diisocyanate homologs having a larger number of rings (polymer MDI), and mixtures of these. Other possible isocyanates are mentioned by way of example in “Kunststoffhandbuch [Plastics handbook], volume 7, Polyurethane [Polyurethanes]”, Carl Hanser Verlag, 3rd edition 1993, chapter 3.2 and 3.3.2.

Suitable polyols used for the production of the polyurethanes of W are selected from the group consisting of polyether polyols, polyester polyols, polyetherester polyols and mixtures thereof.

Polyetherols are by way of example produced from epoxides, for example propylene oxide and/or ethylene oxide, or from tetrahydrofuran with starter compounds exhibiting hydrogen-activity, for example aliphatic alcohols, phenols, amines, carboxylic acids, water, or compounds based on natural substances, for example sucrose, sorbitol or mannitol, with use of a catalyst. Mention may be made here of basic catalysts and double-metal cyanide catalysts, as described by way of example in WO 2006/034800, EP 0090444, or WO 2005/090440.

Polyesterols are by way of example produced from aliphatic or aromatic dicarboxylic acids and polyhydric alcohols, polythioether polyols, polyesteramides, hydroxylated polyacetals, and/or hydroxylated aliphatic polycarbonates, preferably in the presence of an esterification catalyst. Other possible polyols are mentioned by way of example in “Kunststoffhandbuch [Plastics handbook], volume 7, Polyurethane [Polyurethanes]”, Carl Hanser Verlag, 3rd edition 1993, chapter 3.1.

Alternatively, preferably, the one or more polymers contained in the solid material W are one or more polyurethane ureas. According to the present invention, there is no particular restriction as to the polyurethane ureas which are used in the process of the present invention. However, preferably, the polyurethane ureas of W are prepared by a process wherein polyisocyanates are reacted with polyols in the presence of catalysts as well-known in the art. Suitable polyisocyanates and suitable polyols, preferably polyether polyols, are as defined in the foregoing and known in the art.

Alternatively, preferably, the one or more polymers contained in the solid material W are one or more polyisocyanurates. According to the present invention, there is no particular restriction as to the polyisocyanurates which are used in the process of the present invention. However, preferably, the polyisocyanurates of W are prepared by a process wherein polyisocyanates are reacted with polyols in the presence of at least one catalyst as well-known in the art. Suitable polyisocyanates are those listed above. Other possible isocyanates are mentioned by way of example in “Kunststoffhandbuch [Plastics handbook], volume 7, Polyurethane [Polyurethanes]”, Carl Hanser Verlag, 3rd edition 1993, chapter 3.2 and 3.3.2. Preferably, polyisocyanates are tolylene 2,4- and/or 2,6-diisocynate (TDI) or a mixture thereof, monomeric diphenylmethane diisocyanates, and/or diphenylmethane diisocyanate homologs having a larger number of rings (polymer MDI), and mixtures of these. Suitable polyols used for the production of the polyisocyanurates of W are those known in the art, for example those listed “Kunststoffhandbuch [Plastics handbook], volume 7, Polyurethane [Polyurethanes]”, Carl Hanser Verlag, 3rd edition 1993, chapter 3.2 and 3.3.2. Preferably the polyols used for the production of the polyisocyanurates of W are polyester polyol such as a branched polyester polyol based on terephthalic acid and with OH-number of 245 mg KOH/g.

In the context of the present invention, it is also conceivable as a further alternative that preferably the one or more polymers contained in the solid material W are a mixture of one or more of polyurethanes, polyisocyanurates and polyurethane ureas.

Preferably, the reactor unit R_{A} according to (ii) comprises, more preferably consists of, one or more reactors, preferably at least two reactors, more preferably two reactors, wherein more preferably the at least two reactors are arranged in parallel.

Preferably each of the one or more reactors is a heated reactor, an adiabatic reactor or an autoclave.

Preferably each of the one or more reactors is a stirred reactor, preferably a stirred tank reactor.

Preferably each of the one or more reactors has a volume in the range of from 20 to 100 m³, more preferably in the range of from 45 to 55 m³, more preferably in the range of from 48 to 52 m³.

Preferably, the one or more primary amines according to (ii) are free of hydroxyl groups.

Preferably the one or more primary amines used in (ii) are free of hydroxyl groups and free of aldehyde groups. More preferably the one or more primary amines used in (ii) are free of hydroxyl groups, free of aldehyde groups and ketone groups.

Preferably, the atoms forming the one or more primary amines according to (ii) are C, H and N.

Preferably, the one or more primary amines are selected from the group consisting of aliphatic monoamines, aliphatic polyamines, aromatic monoamines, aromatic polyamines, and mixtures of two or more thereof, preferably from the group consisting of aliphatic monoamines, aliphatic polyamines, aromatic monoamines, and mixtures of two or more thereof, more preferably from the group consisting of aliphatic monoamines, aromatic monoamines, and mixtures of two thereof.

More preferably, the one or more primary amines are aliphatic monoamines having the formula H₂NR¹,
wherein R¹ is selected from the group consisting of (C₃-C₂₅) alkyl, phenyl, (C₇-C₂₅) aralkyl, and (C₇-C₂₅) alkaryl, preferably from the group consisting of (C₃-C₂₀) alkyl, (C₈-C₂₂) aralkyl, and (C₈-C₂₂) alkaryl, more preferably from the group consisting of (C₃-C₁₀) alkyl, phenyl, (C₁₀-C₂₀) aralkyl, and (C₁₀-C₂₀) alkaryl. More preferably the aliphatic monoamines are selected from the group consisting of n-butylamines, cyclohexylamines, n-octylamines, n-hexylamines, n-propylamines, n-dodecylamines, n-tridecylamines, n-octadecylamines, and mixtures of two or more thereof, preferably selected from the group consisting of n-butylamines, n-octylamines, n-hexylamines and n-propylamines, wherein more preferably the aliphatic monoamines are n-butylamines

Alternatively, more preferably, the one or more primary amines are aromatic monoamines, wherein the aromatic monoamines more preferably are selected from the group consisting of aniline, toluidine, naphtylamine, and mixtures of two or more thereof, wherein the aromatic monoamines more preferably are aniline.

Alternatively, more preferably, the one or more primary amines are aliphatic polyamines, wherein the aliphatic polyamines more are selected from the group consisting of hexamethylendiamine, ethylenediamine, propanediamine, such as propane-1,3-diamine, propane-1,2-diamine, isophorone diamine, butanediamine, such as butane-1,4-diamine, butane-1,3-diamine, pentadiamine, such as pentane-1,5-diamine, diaminocyclohexane, such as 1,2-diaminocyclohexane, and a mixture of two or more thereof, more preferably selected from the group consisting of hexamethylendiamine, ethylenediamine, propanediamine and butanediamine, more preferably selected from the group consisting of hexamethylendiamine, ethylenediamine, propanediamine; wherein the aliphatic polyamines more preferably are selected from the group consisting of ethylendiamine and propanediamines.

In the context of the present invention, all isomers of the aforementioned aliphatic polyamines can be envisaged. Preferably, propanediamine is propane-1,3-diamine or propane-1,2-diamine.

Alternatively, preferably, the one or more primary amines are aromatic polyamines having the formula H₂N—R²—NH₂,
wherein R² is selected from the group consisting of (C₃-C₂₅) alkylene, (C₆-C₂₅) aralkylene, and (C₆-C₂₅) alkarylene, preferably from the group consisting of (C₈-C₂₂) alkylene, (C₈-C₂₂) aralkylene, and (C₈-C₂₂) alkarylene, more preferably from the group consisting of (C₁₀-C₂₀) alkylene, (C₁₀-C₂₀) aralkylene, and (C₁₀-C₂₀) alkarylene,
wherein the aromatic polyamines are more preferably selected from the group consisting of diaminodiphenylmethane, more preferably 4,4′-diaminodiphenylmethane, phenylenediamine, diaminotoluene, more preferably 2,4-diaminotoluene, and mixtures of two or more thereof.

More preferably, in the context of the present invention, the one or more primary amines are aromatic monoamines or aliphatic monoam-ines. More preferably, the primary amine used in (ii) is aniline or n-butylamine.

Preferably, the ratio of the weight of the solid material W introduced into R_{A} relative to the weight of the one or more primary amines introduced into R_{A} is in the range of from 1:100 to 1:1, more preferably in the range of from 1:40 to 1:3, more preferably in the range of from 1:10 to 1:5.

Preferably, the aminolysis reaction according to (iii) is performed at a temperature in the range of from 50 to 250° C., more preferably in the range of from 80 to 200° C., more preferably in the range of from 100 to 220° C., more preferably in the range of from 140 to 200° C.

Preferably, the aminolysis reaction according to (iii) is performed at a pressure in the range of from 1.0 to 20.0 bar (abs), more preferably in the range of from 1.0 to 20.0 bar (abs), more preferably in the range of from 1.0 to 18.0 bar (abs).

Preferably, the aminolysis reaction according to (iii) is conducted for a duration in the range of from a duration in the range of from 1 to 600 min, more preferably in the range of from 20 to 450 min, more preferably in the range of from 60 to 300 min.

Preferably at most 0.1 weight-%, more preferably from 0 to 0.01 weight-%, more preferably from 0 to 0.001 weight-%, of the mixture M consist of polymer selected from the group consisting of polyurethane, polyurethane urea and polyisocyanurate.

In other words, it is preferred that the mixture M is substantially free of, more preferably free of, polymer selected from the group consisting of polyurethane, and polyisocyanurate, meaning essentially free of, more preferably free of polyurethane, and of polyisocyanurate. Indeed, in the context of the present invention, it is preferred that the aminolysis reaction be complete.

Preferably, the process further comprises, after (iii), removing the mixture M obtained according to (iii) from R_{A}; and
further comprises prior to (v), passing the mixture M removed from R_{A} into a solid-liquid separation unit, obtaining a liquid mixture M_{SLS} comprising the one or more polyurea-containing compounds and the one or more polyols and a solid mixture comprising impurities.

Preferably the impurities being one or more of glass, sand, wood, metals, papers, inorganic solids and polymers other than polyurethanes, polyurethane ureas, polyisocyanurates. The polymers other than polyurethane, polyurethane urea, polyisocyanurate can be for example one or more of polyethylene (PE), polypropylene (PP), polyethylene terephthalate (PET) or polystyrene (PS).

Preferably, the solid-liquid separation unit is a filtration unit or a centrifuge, more preferably a filtration unit, more preferably a filter, more preferably a pocket filter, a bag filter, a membrane filter, a candle filter, an agitated pressure filter, a vacuum belt filter, a frame & plate filter, or a nutsche filter.

Preferably, the solid-liquid separation is performed at a temperature in the range of from 50 to 250° C., more preferably in the range of from 80 to 200° C., more preferably in the range of from 100 to 220° C., more preferably in the range of from 140 to 200° C.

Preferably, the solid-liquid separation is performed at a pressure in the range of from 1.0 to 20.0 bar (abs), more preferably in the range of from 1.0 to 18.0 bar (abs).

Preferably, (iv) comprises
(iv.1) more preferably passing a solvent and the mixture M, more preferably the liquid mixture M_{SLS} as defined in the foregoing, into an evaporation unit EU, obtaining from EU a vapor mixture V comprising the solvent and at least a portion of the one or more primary amines, and a liquid mixture M_{EU} comprising the one or more polyurea-containing compounds and the one or more polyols;
(iv.2) adding a solvent to the mixture M, preferably the liquid mixture M_{SLS} as defined herein, more preferably the liquid mixture M_{EU} obtained according to (iv.1), allowing the one or more polyurea-containing compounds to precipitate, obtaining a mixture comprising one or more precipitated polyurea-containing compounds;
(iv.3) passing the mixture comprising the one or more precipitated polyurea-containing compounds obtained according to (iv.2) through a solid-liquid separation unit SLU, obtaining a liquid mixture P comprising the one or more polyols, and further obtaining a solid mixture U comprising the one or more urea-containing compounds.

Thus, preferably, the present invention relates to a process for recycling one or more polymers selected from the group consisting of polyurethanes, polyurethane ureas, polyisocyanurates, and a mixture of two or more thereof, said one or more polymers being contained in a solid material W, the process comprising
(i) providing the solid material W containing the one or more polymers;
(ii) introducing the solid material W and one or more primary amines in a reactor unit R_{A}, obtaining a mixture, wherein more preferably the atoms forming the one or more primary amines used in (ii) are C, H and N;
(iii) subjecting the mixture obtained according to (ii) in R_{A} to aminolysis reaction conditions, obtaining a mixture M comprising one or more polyurea-containing compounds, and further comprising one or more polyols;
(iv) isolating the one or more polyurea-containing compounds of M obtained according to (iii) from the one or more polyols of M obtained according to (iii), obtaining a mixture U comprising the one or more polyurea-containing compounds and a mixture P comprising the one or more polyols;
wherein (iv) comprises
   (iv.1) more preferably passing a solvent and the mixture M, more preferably the liquid mixture M_{SLS} as defined in the foregoing, into an evaporation unit EU, obtaining from EU a vapor mixture V comprising the solvent and at least a portion of the one or more primary amines, and a liquid mixture M_{EU} comprising the one or more polyurea-containing compounds and the one or more polyols;
   (iv.2) adding a solvent to the mixture M, preferably the liquid mixture M_{SLS} as defined in the present invention, more preferably the liquid mixture M_{EU} obtained according to (iv.1), allowing the one or more polyurea-containing compounds to precipitate, wherein said solvent has a water content in the range of from 0 to 1000 ppm, obtaining a mixture comprising one or more precipitated polyurea-containing compounds;
   (iv.3) passing the mixture comprising the one or more precipitated polyurea-containing compounds obtained according to (iv.2) through a solid-liquid separation unit SLU, obtaining a liquid mixture P comprising the one or more polyols, and further obtaining a solid mixture U comprising the one or more urea-containing compounds;
(v) subjecting the mixture U comprising the one or more polyurea-containing compounds obtained according to (iv) to cleavage reaction conditions in a reactor unit R_{c}, obtaining either one or more corresponding polyamines or one or more corresponding polyisocyanates.

Preferably, the liquid mixture MEU comprises at most 10 weight-%, more preferably at most 8 weight-%, more preferably at most 5 weight-%, of the one or more primary amines, based on the weight of the liquid mixture MEU.

Preferably, the evaporation in EU according to (iv.1) is performed at a pressure difference, being the difference between the pressure before the evaporation unit and the pressure at the unit, being in the range from 0 to 30 bar (abs), preferably in the range of from 0.1 to 25 bar (abs), more preferably in the range of from 0.2 to 20 bar (abs).

Preferably the solvent used in one or more of (iv.1) and (iv.2), more preferably in (iv.1) and (iv.2), has a water content in the range of from 0 to 1000 ppm, more preferably in the range of from 0 to 500 ppm, more preferably in the range of from 0 to 100 ppm. In the context of the present invention, said solvent is thus preferably an anhydrous solvent.

Preferably the solvent used in one or more of (iv.1) and (iv.2), more preferably in (iv.1) and (iv.2), is selected from the group consisting of hydrocarbons, ketones and ethers, more preferably selected from the group consisting of xylene, toluene, n-heptane, methyl ethyl ketone (MEK), dioxane, benzene, heptan-2-one and a mixture of two or more thereof, more preferably is selected from the group consisting of xylene, toluene, n-heptane, benzene and a mixture of two or more thereof, more preferably is xylene or toluene or benzene.

Preferably, the evaporation unit EU used in (iv.1) is one or more of a reactor equipped with a filter, a fractionated distillation column and a flash drum, more preferably a reactor equipped with a filter, more preferably a stirred tank reactor equipped with a filter, such as wire mesh. Optionally, a heat exchanger is located before the evaporation unit EU used in (iv.1), preferably for heating one or more of the solvent and the mixture M, more preferably for heating one or more of the solvent and the liquid mixture M_{SLS} as defined in the foregoing, more preferably for heating the solvent.

Preferably, for (iv.1), the amount of the solvent added is calculated on the basis of the weight ratio of said solvent relative to the one or more polymers of W provided according (i) being in the range of from 0.1:1 to 50:1, more preferably in the range of from 1:1 to 20:1, more preferably in the range of from 1:1 to 1:15, more preferably in the range of from 1:1 to 10:1.

Preferably, isolating according to (iv) comprises,
(iv.1′) optionally a precipitation step, wherein the precipitation step comprises one or more of
   (iv.1′.a) cooling the mixture M obtained from (iii), preferably to a temperature in the range of 15 to 100° C., more preferably in the range of 15 to 30° C., more preferably in the range of 20 to 25° C.;
   (iv.1′.b) evaporating a portion of the one or more primary amines from the mixture M obtained from (iii); and
   (iv.1′.c) adding a solvent to the mixture M obtained from (iii);
      obtaining a mixture UP comprising the mixture U comprising the one or more polyurea-containing compounds and the mixture P comprising the one or more polyols; and
(iv.2′) a separation step, wherein the separation step comprises one or more of
   (iv.2′.a) subjecting the mixture M obtained from (iii) or the mixture UP obtained from (iv.1′) to extraction conditions, obtaining a stationary phase comprising the mixture U comprising the one or more polyurea-containing compounds and a mobile phase comprising the mixture P comprising the one or more polyols,
      wherein the extraction conditions comprise adding a solvent to the mixture M or to the mixture UP; and
(iv.2′.b) filtrating the mixture M obtained from (iii) or the mixture UP obtained from
   (iv.1′), obtaining a filter cake comprising the mixture U comprising the one or more polyurea-containing compounds and a filtrate comprising the mixture P comprising the one or more polyols.

Preferably, the solvent in one or more of (iv.1′.c) and (iv.2′.a), more preferably in (iv.1′.c) and (iv.2′.a), is selected from the group consisting of hydrocarbons, ketones and ethers, more preferably from the group consisting of benzene, xylene, toluene, n-heptane, methyl ethyl ketone (MEK), dioxane, heptan-2-one and a mixture of two or more thereof, more preferably from the group consisting of xylene, toluene, n-heptane, benzene and a mixture of two or more thereof, wherein the solvent more preferably is benzene, xylene or toluene.

In the context of the present invention, the one or more polyols obtained as described above may be employed for every conceivable use in industrial and consumer applications, including, but not limited to, polyurethane compounds which, in turn, may be employed for uses including, but not limited to:
Foam cushioning, for example for furniture, bedding, and packaging
Rigid foam insulation, for example for building and refrigeration
Flexible foam, for example for upholstery, mattrasses, footwear, and seals
Elastomers, for example for automotive and industrial parts, such as suspension bushings, gaskets, and seals
Adhesives, sealants, and coatings
Roller and drive belts, for example for conveyors, power transmissions, and agricultural equipment
Medical devices and implants, such as prosthetics and artificial heart valves
Fibers, non-woven fabrics, and textiles
Paints, coatings, and inks
Electronic components, such as casings, gaskets, and vibration dampers.

Preferably, for (iv.2), the amount of the solvent added is calculated on the basis of the weight ratio of said solvent relative to the one or more polymers of W provided according (i) being in the range of from 0.1:1 to 50:1, more preferably in the range of from 1:1 to 20:1, more preferably in the range of from 1:1 to 15:1, more preferably in the range of from 1:1 to 10:1.

Preferably, the solid-liquid separation unit SLU in (iv.3) is a filter.

Preferably at most 0.1 weight-%, more preferably from 0 to 0.01 weight-%, more preferably from 0 to 0.001 weight-%, of the mixture U consist of polyol.

In other words, it is preferred that the mixture U be substantially free of, more preferably free of, polyol. This can be for example identified with 13C_NMR or HPLC.

Preferably, the process further comprises, after (iv) and prior to (v), washing the mixture U obtained according to (iv) with a solvent on SLU, wherein more preferably the solvent is selected from the group consisting of hydrocarbons, ketones and ethers, more preferably selected from the group consisting of benzene, xylene, toluene, n-heptane, methyl ethyl ketone (MEK), dioxane, heptan-2-one and a mixture of two or more thereof, preferably is selected from the group consisting of xylene, toluene, n-heptane, benzene and a mixture of two or more thereof, more preferably is benzene, xylene or toluene.

Preferably, the ratio of the weight of the solvent relative to the weight of the solid mixture U is in the range of from 0.01:1 to 100:1, preferably in the range of from 0.1:1 to 10:1, more preferably in the range of from 1:1 to 5:1.

Preferably said solvent has a water content in the range of from 0 to 1000 ppm, more preferably in the range of from 0 to 500 ppm, more preferably in the range of from 0 to 100 ppm. In the context of the present invention, said solvent is thus preferably an anhydrous solvent.

Preferably the solvent is the same as used in (iv.1) and (iv.2).

Preferably at most 0.1 weight-%, more preferably from 0 to 0.01 weight-%, more preferably from 0 to 0.001 weight-%, of the mixture U consist of polyol.

In other words, it is preferred that the mixture U be substantially free of, more preferably free of, polyol.

Preferably the reactor unit R_{c} comprises one or more reactors, more preferably at least two reactors, more preferably in the range of from 2 to 5 reactors, more preferably two reactors, the at least two reactors being more preferably arranged in parallel.

Preferably R_{c}, EU and SLU be three distinctive units, wherein EU is located upstream of SLU which is located upstream of R_{c}.

Alternatively, preferably, R_{c} is used for (iv) as one or more of the evaporation unit EU and the solid-liquid separation unit SLU, more preferably as the evaporation unit EU and the solid-liquid separation unit SLU. Hence, more preferably (iv) and (v) are performed in the same unit, said unit be more preferably a stirred tank reactor equipped with a filter.

Preferably, the cleavage reaction conditions according to (v) are hydrolysis reaction conditions. More preferably, (v) comprises
admixing water with the mixture U comprising the one or more polyurea-containing compounds obtained according to (iv) in R_{c}, wherein the weight ratio of water relative to the one or more compounds is in the range of from 1:1 to 50:1, more preferably in the range of from 5:1 to 20:1, more preferably in the range of from 10:1 to 18:1, obtaining one or more corresponding polyamines.

Preferably the hydrolysis is performed at a temperature in the range of from 150 to 350° C., more preferably in the range of from 210 to 290° C., more preferably in the range of from 230 to 270° C.

Preferably, the hydrolysis is performed for a duration in the range of from 0.1 to 15 h, more preferably in the range of from 0.25 to10 h, more preferably in the range of from 0.5 to 8 h.

Preferably, the hydrolysis is performed at a pressure in the range of from 20 to 70 bar (abs), more preferably in the range of from 25 to 65 bar (abs), more preferably in the range of from 30 to 60 bar (abs).

Preferably, the cleavage reaction conditions according to (v) are acidic cleavage conditions. More preferably, (v) comprises
admixing a Bronsted acid, wherein the Bronsted acid is more preferably selected from the group consisting of hydrochloric acid, a sulfonic acid, and a mixture thereof, with the mixture U comprising the one or more polyurea-containing compounds obtained according to (iv) in R_{c}, obtaining one or more corresponding polyisocyanates.

Preferably the sulfonic acid is one or more of methane sulfonic acid, toluene sulfonic acid and trifluromethane sulfonic acid, more preferably methane sulfonic acid.

Preferably, the cleavage reaction conditions according to (v) are basic cleavage conditions. More preferably, (v) comprises
admixing a Bronsted base, wherein the Bronsted base has a pH value in water in the range of from 8 to 12, with the solid mixture U comprising the one or more polyurea-containing compounds obtained according to (iv) in R_{c}, obtaining one or more corresponding polyisocyanates.

Examples of the base for the cleavage reactions are alkali hydroxides, alkaline earth hydroxides, 1,8-Diazabicyclo[5.4.0]undec-7-ene (DBU), 1,4-diazabicyclo[2.2.2]octane (DABCO), triethylamine and primary amines as defined in the foregoing for the aminolysis reaction.

Preferably, the process further comprises purifying the one or more corresponding polyisocyanates obtained in (v), more preferably by distillation or crystallization, more preferably by distillation.

In the context of the present invention, the one or more polyisocyanates obtained as described above may be employed for every conceivable use in industrial and consumer applications, including, but not limited to, polyurethane compounds which, in turn, may be employed for uses including, but not limited to:
Foam cushioning, for example for furniture, bedding, and packaging
Rigid foam insulation, for example for building and refrigeration
Flexible foam, for example for upholstery, mattrasses, footwear, and seals
Elastomers, for example for automotive and industrial parts, such as suspension bushings, gaskets, and seals
Adhesives, sealants, and coatings
Roller and drive belts, for example for conveyors, power transmissions, and agricultural equipment
Medical devices and implants, such as prosthetics and artificial heart valves
Fibers, non-woven fabrics, and textiles
Paints, coatings, and inks
Electronic components, such as casings, gaskets, and vibration dampers.

Preferably, the process further comprises purifying the one or more corresponding polyamines obtained in (v), more preferably by distillation or extraction.

Preferably, the process is a batch, semi-batch or continuous process, more preferably a batch process.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A process for recycling one or more polymers selected from the group consisting of polyurethanes, polyurethane ureas, polyisocyanurates, and a mixture of two or more thereof, said one or more polymers being contained in a solid material W, the process comprising
   (i) providing the solid material W containing the one or more polymers;
   (ii) introducing the solid material W and one or more primary amines in a reactor unit R_{A}, obtaining a mixture;
   (iii) subjecting the mixture obtained according to (ii) in R_{A} to aminolysis reaction conditions, obtaining a mixture M comprising one or more polyurea-containing compounds, and further comprising one or more polyols;
   (iv) isolating the one or more polyurea-containing compounds of M obtained according to (iii) from the one or more polyols of M obtained according to (iii), obtaining a mixture U comprising the one or more polyurea-containing compounds and a mixture P comprising the one or more polyols;
   (v) subjecting the mixture U comprising the one or more polyurea-containing compounds obtained according to (iv) to cleavage reaction conditions in a reactor unit R_{c}, obtaining either one or more corresponding polyamines or one or more corresponding polyisocyanates.
2. The process of embodiment 1, further comprising, prior to (i), subjecting the solid material W to a pre-treatment, preferably a mechanical pre-treatment, wherein more preferably the mechanical pre-treatment comprises one or more of milling, crushing, shredding and cutting, preferably milling or shredding, the solid material W.
3. The process of embodiment 1 or 2, further comprising, prior to (i), preferably after subjecting the solid material W to a pre-treatment as defined in embodiment 2, drying the solid material W, wherein drying is preferably conducted at a temperature in the range of from 40 to 100° C., more preferably in the range of from 50 to 85° C., and wherein more preferably drying is conducted in a gas atmosphere comprising one or more of nitrogen and oxygen, more preferably air.
4. The process of any one of embodiments 1 to 3, wherein the solid material W is a waste solid material.
5. The process of any one of embodiments 1 to 4, wherein the solid material W is one or more of a powder, pieces of foam, pellets, granulates and flakes.
6. The process of any one of embodiments 1 to 5, wherein the one or more polymers contained in the solid material W are one or more polyurethanes.
7. The process of embodiment 6, wherein the one or more polyurethanes are thermosets or elastomers.
8. The process of any one of embodiments 1 to 5, wherein the one or more polymers contained in the solid material W are one or more polyurethane ureas.
9. The process of any one of embodiments 1 to 5, wherein the one or more polymers contained in the solid material W are one or more polyisocyanurates.
10. The process of any one of embodiments 1 to 9, wherein the reactor unit R_{A} according to (ii) comprises, preferably consists of, one or more reactors, preferably at least two reactors, more preferably two reactors, wherein more preferably the at least two reactors are arranged in parallel.
11. The process of embodiment 10, wherein each of the one or more reactors is a heated reactor, an adiabatic reactor or an auto-clave.
12. The process of embodiment 10 or 11, wherein each of the one or more reactors is a stirred reactor, preferably a stirred tank reactor.
13. The process of any one of embodiments 10 to 12, wherein each of the one or more reactors has a volume in the range of from 20 to 100 m³, preferably in the range of from 45 to 55 m³, more preferably in the range of from 48 to 52 m³.
14. The process of any one of embodiments 1 to 13, wherein the one or more primary amines according to (ii) are free of hydroxyl groups.
15. The process of any one of embodiments 1 to 14, wherein the atoms forming the one or more primary amines according to (ii) are C, H and N.
16. The process of any one of embodiments 1 to 15, wherein the one or more primary amines are selected from the group consisting of aliphatic monoamines, aliphatic polyamines, aromatic monoamines, aromatic polyamines, and mixtures of two or more thereof, preferably from the group consisting of aliphatic monoamines, aliphatic polyamines, aromatic monoamines, and mixtures of two or more thereof, more preferably from the group consisting of aliphatic monoamines, aromatic monoamines, and mixtures of two thereof.
17. The process of embodiment 16, wherein the one or more primary amines are aliphatic monoamines having the formula H₂NR¹,
wherein R¹ is selected from the group consisting of (C₃-C₂₅) alkyl, phenyl, (C₇-C₂₅) aralkyl, and (C₇-C₂₅) alkaryl, preferably from the group consisting of (C₃-C₂₀) alkyl, (C₈-C₂₂) aralkyl, and (C₈-C₂₂) alkaryl, more preferably from the group consisting of (C₃-C₁₀) alkyl, phenyl, (C₁₀-C₂₀) aralkyl, and (C₁₀-C₂₀) alkaryl.
18. The process of embodiment 17, wherein the aliphatic monoamines are selected from the group consisting of n-butylamines, cyclohexylamines, n-octylamines, n-hexylamines, npropylamines, n-dodecylamines, n-tridecylamines, n-octadecylamines, and mixtures of two or more thereof, preferably selected from the group consisting of n-butylamines, n-octylamines, n-hexylamines and n-propylamines, wherein more preferably the aliphatic monoamines are n-butylamines
19. The process of embodiment 16, wherein the one or more primary amines are aromatic monoamines, wherein the aromatic monoamines are selected from the group consisting of aniline, toluidine, naphtylamine, and mixtures of two or more thereof, wherein the aromatic monoamines preferably are aniline.
20. The process of embodiment 16, wherein the one or more primary amines are aliphatic polyamines, wherein preferably the aliphatic polyamines are selected from the group consisting of hexamethylendiamine, ethylenediamine, propanediamine, isophorone diamine, butanediamine, pentadiamine, diaminocyclohexane and a mixture of two or more thereof, more preferably selected from the group consisting of hexamethylendiamine, ethylenediamine, propanediamine and butanediamine, more preferably selected from the group consisting of hexamethylendiamine, ethylenediamine, propanediamine; wherein the aliphatic polyamines more preferably are selected from the group consisting of ethylendiamine and propanediamines.
21. The process of embodiment 16, wherein the one or more primary amines are aromatic polyamines having the formula H₂N—R²—NH₂,
wherein R² is selected from the group consisting of (C₃-C₂₅) alkylene, (C₆-C₂₅) aralkylene, and (C₆-C₂₅) alkarylene, preferably from the group consisting of (C₈-C₂₂) alkylene, (C₈-C₂₂) aralkylene, and (C₈-C₂₂) alkarylene, more preferably from the group consisting of (C₁₀-C₂₀) alkylene, (C₁₀-C₂₀) aralkylene, and (C₁₀-C₂₀) alkarylene,
wherein the aromatic polyamines are more preferably selected from the group consisting of diaminodiphenylmethane, more preferably 4,4′-diaminodiphenylmethane, phenylenediamine, diaminotoluene, more preferably 2,4-diaminotoluene, and mixtures of two or more thereof.
22. The process of any one of embodiments 1 to 21, wherein the ratio of the weight of the solid material W introduced into R_{A} relative to the weight of the one or more primary amines introduced into R_{A} is in the range of from 1:100 to 1:1, preferably in the range of from 1:40 to 1:3, more preferably in the range of from 1:10 to 1:5.
23. The process of any one of embodiments 1 to 22, wherein the aminolysis reaction according to (iii) is performed at a temperature in the range of from 50 to 250° C., preferably in the range of from 80 to 200° C., more preferably in the range of from 100 to 220° C., more preferably in the range of from 140 to 200° C.
24. The process of any one of embodiments 1 to 23, wherein the aminolysis reaction according to (iii) is performed at a pressure in the range of from 1.0 to 20.0 bar (abs), preferably in the range of from 1.0 to 20.0 bar (abs), more preferably in the range of from 1.0 to 18.0 bar (abs).
25. The process of any one of embodiments 1 to 24, wherein the aminolysis reaction according to (iii) is conducted for a duration in the range of from a duration in the range of from 1 to 600 min, preferably in the range of from 20 to 450 min, more preferably in the range of from 60 to 300 min.
26. The process of any one of embodiments 1 to 25, wherein at most 0.1 weight-%, preferably from 0 to 0.01 weight-%, more preferably from 0 to 0.001 weight-%, of the mixture M consist of polymer selected from the group consisting of polyurethane, polyurethane urea and polyisocyanurate.
27. The process of any one of embodiments 1 to 26, further comprising, after (iii), removing the mixture M obtained according to (iii) from R_{A}; and further comprising prior to (v), passing the mixture M removed from R_{A} into a solid-liquid separation unit, obtaining a liquid mixture M_{SLS} comprising the one or more polyurea-containing compounds and the one or more polyols and a solid mixture comprising impurities.
28. The process of embodiment 27, wherein the solid-liquid separation is performed at a temperature in the range of from 50 to 250° C., preferably in the range of from 80 to 200° C., more preferably in the range of from 100 to 220° C., more preferably in the range of from 140 to 200° C.;
wherein, preferably, the solid-liquid separation is performed at a pressure in the range of from 1.0 to 20.0 bar (abs), more preferably in the range of from 1.0 to 18.0 bar (abs).
29. The process of any one of embodiments 1 to 28, wherein (iv) comprises
   (iv.1) preferably passing a solvent and the mixture M, more preferably the liquid mixture M_{SLS} as defined in embodiment 27, into an evaporation unit EU, obtaining from EU a vapor mixture V comprising the solvent and at least a portion of the one or more primary amines, and a liquid mixture Meu comprising the one or more polyurea-containing compounds and the one or more polyols;
   (iv.2) adding a solvent to the mixture M, preferably the liquid mixture M_{SLS} as defined in embodiment 27, more preferably the liquid mixture M_{EU} obtained according to (iv.1), allowing the one or more polyurea-containing compounds to precipitate, obtaining a mixture comprising one or more precipitated polyurea-containing compounds;
   (iv.3) passing the mixture comprising the one or more precipitated polyurea-containing compounds obtained according to (iv.2) through a solid-liquid separation unit SLU, obtaining a liquid mixture P comprising the one or more polyols, and further obtaining a solid mixture U comprising the one or more urea-containing compounds.
30. The process of embodiment 29, wherein the solvent used in one or more of (iv.1) and (iv.2), preferably in (iv.1) and (iv.2), is selected from the group consisting of hydrocarbons, ketones and ethers, more preferably selected from the group consisting of xylene, toluene, n-heptane, methyl ethyl ketone (MEK), dioxane, benzene, heptan-2-one and a mixture of two or more thereof, more preferably is selected from the group consisting of xylene, toluene, n-heptane, benzene and a mixture of two or more thereof, more preferably is xylene or toluene or benzene.
31. The process of embodiment 29 or 30, wherein the evaporation unit EU used in (iv.1) is one or more of a reactor equipped with a filter, a fractionated distillation column and a flash drum, more preferably a reactor equipped with a filter, more preferably a stirred tank reactor equipped with a filter, wherein a heat exchanger is optionally located before the evaporation unit EU used in (iv.1), preferably for heating one or more of the solvent and the mixture M, more preferably for heating one or more of the solvent and the liquid mixture M_{SLS} as defined in the foregoing, more preferably for heating the solvent.
32. The process of any one of embodiments 29 to 31, wherein for (iv.1), the amount of the solvent added is calculated on the basis of the weight ratio of said solvent relative to the one or more polymers of W provided according (i) being in the range of from 0.1:1 to 50:1, preferably in the range of from 1:1 to 20:1, more preferably in the range of from 1:1 to 1:15, more preferably in the range of from 1:1 to 10:1.
33. The process of any one of embodiments 29 to 32, wherein for (iv.2), the amount of the solvent added is calculated on the basis of the weight ratio of said solvent relative to the one or more polymers of W provided according (i) being in the range of from 0.1:1 to 50:1, preferably in the range of from 1:1 to 20:1, more preferably in the range of from 1:1 to 15:1, more preferably in the range of from 1:1 to 10:1.
34. The process of any one of embodiments 1 to 28, wherein isolating according to (iv) comprises,
   (iv.1′) optionally a precipitation step, wherein the precipitation step comprises one or more of
      (iv.1′.a) cooling the mixture M obtained from (iii), preferably to a temperature in the range of 15 to 100° C., preferably in the range of 15 to 30° C., more preferably in the range of 20 to 25° C.;
      (iv.1′.b) evaporating a portion of the one or more primary amines from the mixture M obtained from (iii); and
      (iv.1′.c) adding a solvent to the mixture M obtained from (iii);
      obtaining a mixture UP comprising the mixture U comprising the one or more polyurea-containing compounds and the mixture P comprising the one or more polyols; and
(iv.2′) a separation step, wherein the separation step comprises one or more of
   (iv.2′.a) subjecting the mixture M obtained from (iii) or the mixture UP obtained from (iv.1′) to extraction conditions, obtaining a stationary phase comprising the mixture U comprising the one or more polyurea-containing compounds and a mobile phase comprising the mixture P comprising the one or more polyols,
   wherein the extraction conditions comprise adding a solvent to the mixture M or to the mixture UP; and
   (iv.2′.b) filtrating the mixture M obtained from (iii) or the mixture UP obtained from (iv.1′), obtaining a filter cake comprising the mixture U comprising the one or more polyurea-containing compounds and a filtrate comprising the mixture P comprising the one or more polyols.
35. The process of claim 34, wherein the solvent in one or more of (iv.1′.c) and (iv.2′.a), preferably in (iv.1′.c) and (iv.2′.a), is selected from the group consisting of hydrocarbons, ketones and ethers, more preferably from the group consisting of benzene, xylene, toluene, n-heptane, methyl ethyl ketone (MEK), dioxane, heptan-2-one and a mixture of two or more thereof, more preferably selected from the group consisting of xylene, toluene, nheptane, benzene and a mixture of two or more thereof, wherein the solvent more preferably is benzene, xylene or toluene.
36. The process of any one of embodiments 1 to 35, further comprising, after (iv) and prior to (v), washing the mixture U obtained according to (iv) with a solvent on SLU, wherein the solvent is selected from the group consisting of hydrocarbons, ketones and ethers, more preferably selected from the group consisting of benzene, xylene, toluene, n-heptane, methyl ethyl ketone (MEK), dioxane, heptan-2-one and a mixture of two or more thereof, more preferably is selected from the group consisting of xylene, toluene, n-heptane, benzene and a mixture of two or more thereof, more preferably is benzene, xylene or toluene; wherein the ratio of the weight of the solvent relative to the weight of the solid mixture U is in the range of from 0.01:1 to 100:1, more preferably in the range of from 0.1:1 to 10:1, more preferably in the range of from 1:1 to 5:1.
37. The process of embodiment 36, wherein the solvent has a water content in the range of from 0 to 1000 ppm, preferably in the range of from 0 to 500 ppm, more preferably in the range of from 0 to 100 ppm, wherein the solvent more preferably is an anhydrous solvent.
38. The process of any one of embodiments 1 to 37, wherein at most 0.1 weight-%, preferably from 0 to 0.01 weight-%, more preferably from 0 to 0.001 weight-%, of the mixture U consist of polyol.
39. The process of any one of embodiments 1 to 38, wherein the cleavage reaction conditions according to (v) are hydrolysis reaction conditions.
40. The process of embodiment 39, wherein (v) comprises admixing water with the mixture U comprising the one or more polyurea-containing compounds obtained according to (iv) in R_{c}, wherein the weight ratio of water relative to the one or more polyurea-containing compounds is in the range of from 1:1 to 50:1, preferably in the range of from 5:1 to 20:1, more preferably in the range of from 10:1 to 18:1, obtaining one or more corresponding polyamines.
41. The process of embodiment 39 or 40, wherein the hydrolysis is performed at a temperature in the range of from 150 to 350° C., preferably in the range of from 210 to 290° C., more preferably in the range of from 230 to 270° C.;
wherein preferably the hydrolysis is performed for a duration in the range of from 0.1 to 15 h, more preferably in the range of from 0.25 to10 h, more preferably in the range of from 0.5 to 8 h.
42. The process of any one of embodiments 39 to 41, wherein the hydrolysis is performed at a pressure in the range of from 20 to 70 bar (abs), preferably in the range of from 25 to 65 bar (abs), more preferably in the range of from 30 to 60 bar (abs).
43. The process of any one of embodiments 1 to 38, wherein the cleavage reaction conditions according to (v) are acidic cleavage conditions.
44. The process of embodiment 43, wherein (v) comprises admixing a Bronsted acid, wherein the Bronsted acid is preferably selected from the group consisting of hydrochloric acid, a sulfonic acid, and a mixture thereof, with the mixture U comprising the one or more polyurea-containing compounds obtained according to (iv) in R_{c}, obtaining one or more corresponding polyisocyanates.
45. The process of any one of embodiments 1 to 38, wherein the cleavage reaction conditions according to (v) are basic cleavage conditions.
46. The process of embodiment 45, wherein (v) comprises admixing a Bronsted base, wherein the Bronsted base has a pH value in water in the range of from 8 to 12, with the solid mixture U comprising the one or more polyurea-containing compounds obtained according to (iv) in R_{c}, obtaining one or more corresponding polyisocyanates.
47. The process of any one of embodiments 1 to 38 and 43 to 46, further comprising purifying the one or more corresponding polyisocyanates obtained in (v), preferably by distillation or crystallization, more preferably by distillation.
48. The process of any one of claims 1 to 42, further comprising purifying the one or more corresponding polyamines obtained in (v), preferably by distillation or extraction.
49. The process of any one of embodiments 1 to 48, being a batch, semi-batch or continuous process, preferably a batch process.

In the context of the present invention, a polyurethane can be abbreviated PUR or PU, referring to a class of polymers comprising carbamate (urethane) links. Further, a polyisocyanurate can be abbreviated as PIR, referring to a polymer comprising isocyanurate groups.

In the context of the present invention, ureas are organic compounds with the formula (RR′N)₂CO, wherein R and R′ independently from one another is hydrogen or an organic residue, for example an alkyl residue or an aryl residue. Thus, this definition includes the specific chemical compound ((H₂N)₂CO).

Further, in the context of the present invention, PMDI relates to polymeric diphenylmethane diisocyanate, also known as technical MDI, being a mixture of methylenediphenyl diisocyanates and homologous aromatic polyisocyanates. Thus, it is a mixture of compounds with several (typically up to 6) phenylene groups, each of which carrying an isocyanate group.

Furthermore in the context of the present invention, the term “polyurea-containing compound” refers to a compound, preferably a polymer, comprising at least two urea groups. In particular, a polyurea-containing compound, preferably a polyurea-containing polymer, in the context of the present invention is preferably a compound derived from a polyurethane which was subjected to aminolysis.

In the context of the present invention, the term “primary amine” encompasses “primary monoamine” and “primary polyamine”. As well-known in the art, primary amine refers to compound having one or more amino groups, wherein the nitrogen atom is directly bond with only one C atom.

In the context of the present invention, an alkyl group consists of carbon atoms and hydrogen atoms.

The present invention is further illustrated by the following examples.

### EXAMPLES

### Reference Example 1: Determination of FTIR Spectra

IR spectroscopy was carried out to determine the conversion of the amination and aminolysis reaction.

For urea synthesis, the absence of the NCO-stretching vibration at 2200-2400 cm⁻¹ indicates complete conversion of a respective isocyanate component (e.g. M20S).

For aminolysis, the area ratio of the urethane vibration (1700-1750 cm⁻¹) before and after the reaction correlates to the conversion.

For solubility tests, the characteristic feature to evaluate the solubility of a polyurea was the presence of the characteristic carbonyl vibration (1600-1700 cm-1) in the sample phase. The area of this vibration correlates to the mass fraction of the urea. For determining the carbonyl vibration as characteristic feature for the solubility of urea, the IR apparatus was calibrated with a 1 weight-% solution of dibutylamine and a n-butylamine.

### Reference Example 2: Determination of 13C-NMR Spectra

To evaluate the purity of a synthesized urea ¹³C-NMR spectroscopy was carried out. The absence of peaks in the range of 50 until 80 ppm chemical shift indicates the absence of polyols. The sample was solved in DMSO. As reference standard tetramethylsilane was used.

### Reference Example 3: Appliance and Construction Foams and their Compositions

a) PUR-appliance foam: A model foam was prepared by mixing 56 weight-% Lupranat® M20S, (Lupranat® M20S is a commercial product of BASF, having a typical NCO content of 31.5 g/100 g and a viscosity of about 200 mPas at 25° C.), and 35 weight-% polyols, a mixture of polyols 1, 2 and 3, containing additionally the additives required for foaming, e.g. catalysts, surfactants, blowing agents
b) PIR-construction foam: A model foam was prepared by mixing 71.4 weight-% Lupranat® M50S, (Lupranat® M50S is a commercial product of BASF, having a typical NCO content of 31.5 g/100 g and a viscosity of about 550 mPas at 25° C.), and 18.4 weight-% polyols, polyols 4 being the main polyol, including additionally additives required for foaming, e.g. catalysts, surfactants, blowing agents;
c) Polyols (see FIG. 2):
   Polyol 1 is a polyether-polyol synthesized from vicinal-TDA (toluenediamine) as starter and PEO and PPO side chains having a OH-number of 160 mg KOH/g
   Polyol 2 is a polyether-polyol synthesized from vicinal-TDA (toluenediamine) as starter and PPO side chains; having a OH-number of 400 mg KOH/g
   Polyol 3 is a mixture of two polyether-polyols, wherein one polyether-polyol is synthesized from sorbitol as starter and PPO side chains and the other polyether-polyol is synthesized from glycerol as starter and PPO side chains having a OH-number of 430 mg KOH/
   Polyol 4 is a slightly branched polyester polyol based on terephthalic acid and with OH-number of 245 mg KOH/g)
d) Surfactants: silicone oils;
e) Blowing agents: water, cyclopentane;
f) Catalysts: Lupragen® N100, Lupragen® N206, Lupragen® N600 (all commercially available from BASF); Tese catalysts are usually tertiary amines with the general formular R₁R₂R₃N. The fragments R₁ R₂ R₃ are alkyl side chains with a chain length of 3-10 carbon atoms.

### Reference Example 4: End-of-Life Foam

a) PUR-appliance end-of-life (EoL) foam: foam powder (MDI-based) obtained from a refrigerator dismantling company
b) PU-flexible conventional foam from a mattress dismantling company TDI-based);
c) Polyols:

| Polyol type | Type | Composition |
|---|---|---|
| Polyether-polyol | Sugar | Sorbitol-PPO |
| Polyether-polyol | TDA | TDA-PPO, TDA-PPO-PEO |
| Polyether-polyol | Amine | Ethylenediamine-PPO |
| Polyether-polyol | Soft | Gly-PPO |
| Polyester-polyol | Ester | Phthalic acid/Diethylenglycol (DEG) |
| Polyether-/polyester- | Hybrid | Biodiesel/Sucrose |
| polyol | | |
| Polyether-polyol | MDA | MDA-PPO |

### Reference Example 5: Solubility Tests

Four different model systems were prepared for conducting solubility tests of the respective resulting urea in presence of polyether/polyester-polyols.

### 5.1. Synthesis of Model Systems

5.1.a Polyurea Derived from PMDI (Using a Secondary Amine)

In a 2L round flask 50 g water-free dibutylamine (DBA) and 300 g monochlorobenzene (MCB) were mixed and stirred continuously. Via a dropping funnel a solution consisting of 17 g polymeric isocyanate (Lupranat® M20S having a typical NCO content of 31.5 g/100 g and a viscosity of about 200 mPas at 25° C.; BASF) and 281 g monochlorobenzene was added dropwise over a time period of 1 h. The reaction temperature was increased from 22.7 to 30.1° C. during the reaction. Afterwards, the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. Monochlorobenzene and excess dibutylamine were evaporated under vacuum and a highly viscous residue was obtained. The yield was 33.9 g corresponding to 98%.

5.1.b Polyurea Derived from PMDI (Using a Primary Amine)

In a 2L round flask 50 g water-free n-octylamine and 450 g monochlorobenzene were mixed and stirred continuously. Via a dropping funnel a solution consisting of 16 g polymeric isocyanate (Lupranat® M20S having a typical NCO content of 31.5 g/100 g and a viscosity of about 200 mPas at 25° C.; BASF) and 307 g monochlorobenzene was added dropwise over a time period of 1 h. The reaction temperature was increased from 22.7 to 50.8° C. during the reaction. Afterwards the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. Monochlorobenzene and excess n-octylamine were evaporated under vacuum and a crystalline solid was obtained. The yield was 32.0 g corresponding to 98%.

5.1.c Polyurea Derived from PMDI (Using a Primary Amine)

In a 2L round flask 41 g water-free n-butylamine and 364 g monochlorobenzene were mixed and stirred continuously. Via a dropping funnel a solution consisting of 22 g polymeric isocyanate (Lupranat® M20S having a typical NCO content of 31.5 g/100 g and a viscosity of about 200 mPas at 25° C.; BASF) and 406 g monochlorobenzene was added dropwise over a time period of 1 h. The reaction temperature was increased from 21.7 to 31.4° C. during the reaction. Afterwards the reaction mixture was stirred overnight. The obtained product was analyzed using FTIR-spectroscopy. The absence the NCO-stretching vibration in the range of from 2200-2400 cm⁻¹ indicated complete conversion of the isocyanate groups. The monochlorobenzene and excess n-butylamine were evaporated under vacuum and a crystalline solid was obtained. The yield was 34.1 g corresponding to 98%.

5.1.d Polyurea Derived from PMDI (Using a Primary Amine)

In a 2L round flask 84 g water-free aniline and 762 g monochlorobenzene were mixed and stirred continuously. Via a dropping funnel a solution consisting of 36 g polymeric isocyanate (Lupranat® M20S having a typical NCO content of 31.5 g/100 g and a viscosity of about 200 mPas at 25° C.; BASF) and 652 g monochlorobenzene was added dropwise over a time period of 1 h. Afterwards the reaction mixture was stirred overnight. The obtained product was analyzed using FTIR-spectroscopy. The absence of the NCO-stretching vibration in the range of from 2200-2400 cm⁻¹ indicated complete conversion of the isocyanate groups. The monochlorobenzene and excess aniline was evaporated under vacuum and a crystalline solid was obtained. The yield was 61.2 g corresponding to 98%.

5.1.e Polyurea Derived from PMDI (Using a Primary Amine)

In a 2 L round flask 33.1 g water-free toluenediamine (TDA-aromatic polyamine) and 1100 g monochlorobenzene were mixed and stirred continuously. Via a dropping funnel a solution consisting of 3.6 g polymeric isocyanate (Lupranat® M20S having a typical NCO content of 31.5 g/100 g and a viscosity of about 200 mPas at 25° C.; purchased from BASF) and monochlorobenzene (12 g) was added dropwise within 1 h. Afterwards the reaction mixture was stirred overnight. The obtained product was analyzed using FTIR-spectroscopy. The absence of the NCO-stretching vibration at 2200-2400 cm⁻¹ indicated complete conversion of the isocyanate groups. The monochlorobenzene and excess TDA was evaporated under vacuum and a crystalline solid (6.9 g) was obtained. The yield was 99%.

5.1.f Polyurea Derived from PMDI (Using a Primary Amine)

In a 2 L round flask 33 g water-free ethylenediamine (aliphatic polyamine) and 300 g monochlorobenzene were mixed and stirred continuously. Via a dropping funnel a solution consisting of 22 g polymeric isocyanate (Lupranat® M20S having a typical NCO content of 31.5 g/100 g and a viscosity of about 200 mPas at 25° C.; purchased from BASF) and monochlorobenzene (340 g) was added dropwise within 1 h. Afterwards the reaction mixture was stirred overnight. The obtained product was analyzed using FTIR-spectroscopy. The absence of the NCO-stretching vibration at 2200-2400 cm⁻¹ indicated complete conversion of the isocyanate groups. The monochlorobenzene and excess ethylenediamine was evaporated under vacuum and a crystalline solid (29.8 g) was obtained. The yield was 93%.

### 5.2. Evaluation of Polyurea Solubility

### 5.2.a Solubility Screening

Approximately 5 g of a polyurea according to a respective model system, 2.5 g polyether-polyol-mixture, containing TDA, glycerol and sucrose derived polyether-polyols and 25 g of a respective solvent were mixed at room temperature. After 90 min stirring the polyether-polyols were added and an IR sample was taken, diluted with chloroform, and analyzed. The area of the carbonyl vibration in the range of from 1600-1700 cm⁻¹ correlates with the solved amount of urea. The detection limit was <1.0 weight-%. The results for the solubility tests for the model systems prepared according to 1.a-f are shown in table 1 below.

TABLE 1
Results for the solubility tests for the model systems prepared according to 5.1.a-f.

| | Polyurea | Polyurea | Polyurea | Polyurea | | |
|---|---|---|---|---|---|---|
| | acc. to Ref. | acc. to Ref. | acc. to Ref. | acc. to Ref. | | |
| | Ex. 5.1.a | Ex. 5.1.c | Ex. 5.1.b | Ex. 5.1.d | Polyurea | Polyurea |
| | (secondary | (primary | (primary | (primary | acc. to | acc. to |
| | amine) | amine) | amine) | amine) | Ref. Ex. | Ref. Ex. |
| Solvent | [wt.-%] | [wt.-%] | [wt.-%] | [wt.-%] | 5.1.e | 5.1.f |
| Methyl-tert.- | 8.8 | <1.0 | <1.0 | <1.0 | <1.0 | <1.0 |
| butylether | | | | | | |
| (MTBE) | | | | | | |
| Methanol | — | <1.0 | <1.0 | <1.0 | <1.0 | <1.0 |
| Ethanol | 10.1 | — | — | — | — | — |
| Cyclohexane | 2.2 | <1.0 | <1.0 | <1.0 | <1.0 | <1.0 |
| Water | 12.3 | <1.0 | <1.0 | <1.0 | <1.0 | <1.0 |
| Benzene | — | <1.0 | <1.0 | <1.0 | <1.0 | <1.0 |
| Toluene | 9.1 | — | — | — | — | — |
| Xylene | — | <1.0 | <1.0 | <1.0 | <1.0 | <1.0 |

The results of the solubility tests (table 1), show that the polyurea derived from the reaction of a secondary amine, in particular dibutylamine, with PMDI was soluble in the tested solvents whereas the polyureas derived from the reaction of a primary amine with PMDI were practically not soluble. In particular, it was found that 9.1 weight-% of the polyurea derived from the reaction of dibutylamine with PMDI could be dissolved in toluene. It can thus be expected that comparable solubility would be achieved in xylene. In contrast thereto, less than 1.0 weight-% of the polyurea derived from the reaction of a primary amine, in particular n-octylamine, n-butylamine and aniline as used in Reference Examples 1.b-1.d, were soluble in xylene. It is thus considered that the polyreas of these examples are not soluble in the tested solvents.

### Example 1: Aminolysis of PUR-Appliance Foam with n-Butylamine

30 g of the PUR-appliance foam according to Reference Example 3 a) was milled using a coffee grinder and dried overnight at 100° C. in a nitrogen flushed atmosphere under atmospheric pressure. Afterwards the dried foam and 150 g n-butylamine were charged into a stirred autoclave. The aminolysis reaction was conducted at 140° C. and approximately 5 bar (depending on the vapor pressure of n-butylamine) for 75 min. After the reaction the mixture was cooled down to room temperature and approximately 120 mL of n-butylamine were evaporated. To the resulting residue 150 g xylene were added and the precipitation of the polyurea started. To complete the precipitation, the remaining n-butylamine was evaporated. Subsequently the precipitated polyurea was filtered off and washed several times with xylene. With the aid of ¹³C-NMR- and IR-spectroscopy the conversion and the purity of the precipitate were documented. As can be seen from the IR spectra shown in FIG. 1 no characteristic urethane vibration was detected for the polyurea. The conversion was about 95% and only small amounts of ether groups could be detected, as can be seen from the 13C-NMR spectrum shown in FIG. 2. The purity was higher than 90%. 16.1 g of the corresponding polyurea-containing compound was obtained (yield: 61%).

### Example 2: Aminolysis of PUR-Appliance Foam with Aniline

10 g of the PUR-appliance foam according to Reference Example 3 a) was milled using a coffee grinder and dried overnight at 100° C. in a nitrogen flushed atmosphere under atmospheric pressure. Afterwards the dried foam and 110 g aniline were charged into a 0.5 L round flask. The aminolysis reaction was conducted at 180° C. under reflux for 1 h. During the aminolysis reaction all compounds were completely dissolved in aniline. After the reaction the mixture was cooled down to room temperature and the formed polyurea started precipitating. After 12 h the precipitate was filtered off. The solids were washed several times with xylene and dried overnight, yielding 3.5 g polyurea corresponding to 40%. Excess aniline was evaporated from the filtrate, and 250 g xylene was added to the residue. The solution was stirred overnight. The newly formed precipitate was filtrated, washed and dried, yielding 2.1 g polyurea corresponding to 24%. With the aid of 13C-NMR- and IR-spectroscopy the aminolysis conversion and the purity of the precipitate were documented. The conversion was about 98% and no ether groups be detected. The purity was higher than 95%. 5.6 g of the corresponding polyurea-containing compound was obtained (yield: 64%).

### Example 3: Aminolysis of PUR-Appliance End-of-Life (EoL) Foam with n-Butylamine

24 g of the PUR-appliance end-of-life (EoL) foam according to Reference Example 4 a) and 120 g n-butylamine were charged into a stirred autoclave. The aminolysis reaction was conducted at 140° C. and approximately 5 bar (depending on the vapor pressure of n-butylamine) for 75 min. After the reaction the mixture was cooled down to room temperature. After pressure relaxation the mixture was heated to 60° C. and filtered over a nylon filter to remove insoluble inorganic impurities and organic impurities (mᵢₘₚᵤᵣᵢₜᵢₑₛ=2.0 g). Then, approximately 120 mL of n-butylamine were evaporated. To the resulting residue 150 g xylene were added and after some time the precipitation of the polyurea started. To complete the precipitation, the remaining n-butylamine was evaporated. Subsequently the precipitated polyurea was filtered off and washed several times with xylene. The aminolysis conversion and the purity of the precipitate were analyzed using ¹³C-NMR- and IR-spectroscopy. The conversion was about 98% and no ether groups could be detected. The purity was higher than 95%. 8.6 g of the corresponding polyurea-containing compound was obtained.

### Example 4: Aminolysis of PUR-Appliance End-of-Life (EoL) Foam with Aniline

25 g of the PUR-appliance end-of-life (EoL) foam according to Reference Example 4 a) and 250 g aniline were added to a 0.5 L round flask. The aminolysis reaction was conducted at 180° C. under reflux for 1 h. During the aminolysis reaction inorganic impurities precipitated. After the reaction the mixture was filtrated using a nylon filter and the filtrate was cooled down to room temperature (mᵢₘₚᵤᵣᵢₜᵢₑₛ=2.1 g). Then, the formed polyurea started to precipitate. After 12 h the precipitate was filtered off. The solids were washed several times with xylene and dried overnight (m_{polyurea,1}=10.3 g). Excess aniline was evaporated from the filtrate, and 250 g xylene were added to the residue. The solution was stirred overnight. The resulting precipitate was filtered off, washed, and dried. (m_{polyurea,2}=1.8 g, Yield=24%). Using ¹³C-NMR- and IR-spectroscopy the conversion and purity of the precipitate were analyzed. The conversion was about 90% and no ether groups could be detected. The purity was higher than 95%, 12.1 g of the corresponding polyurea-containing compound was obtained.

### Example 5: Aminolysis of PU Flexible Foam with n-Butylamine

15 g of the PU flexible foam according to Reference Example 4 b) was mixed with liquid nitrogen, then milled using a coffee grinder and dried overnight at 100° C. in a nitrogen flushed atmosphere under atmospheric pressure. Afterwards the dried foam and 150 g of n-butylamine were added to a stirred autoclave. The aminolysis reaction was conducted at 140° C. and approximately 5 bar (depending on the vapor pressure of n-butylamine) for 75 min. After the reaction the mixture was filtered at 140° C. under pressure to remove solid impurities. Then, n-butylamine was evaporated under vacuum and the remaining residue washed with n-heptane several times. Using ¹³C-NMR- and IR-spectroscopy the conversion and purity of the precipitated polyurea were analyzed. The conversion was about 90% and no ether groups could be detected. The purity was higher than 80%. 6.1 g of the corresponding polyurea-containing compound was obtained.

### Example 6: Aminolysis of a Polyisocyanurate (PIR) Construction Foam with Aniline

15 g of the dried and shredded PIR-construction foam according to Reference Example 3 b) a polyisocyanurate construction foam and 400 g aniline were charged into a round flask equipped with a reflux condenser. The aminolysis reaction was conducted at 180° C. for 60 min until the foam components were completely dissolved in aniline. The mixture was cooled down to room temperature and the polyurea started precipitating. The precipitated polyurea was filtered, and the filtrate was concentrated by evaporative removal of aniline. The remaining solids were also filtered off and washed several times with acetone. Using ¹³C-NMR- and IR-spectroscopy the conversion and purity of the precipitate were analyzed. The conversion was about 90% and no ether groups could be detected. The purity was higher than 95%, 12.2 g of the corresponding polyurea-containing compound was obtained (yield: 60%).

### Example 7: Hydrolysis of Polyurea-Containing Compound (Polymer) with Water

5.1 g of polyurea obtained from Example 3 and 75 g water were charged into a pressure autoclave. The mixture was heated to 250° C. and the temperature was hold for 5 h, whereby the pressure was approximately 40 bar (abs). After cooling down the mixture was analyzed using GC to evaluate the amount of PMDA. The purity was determined based on the area ratios of the GC peaks, accordingly the purity was 85% (m_{residue}=4.3 g).

### BRIEF DESCRIPTION OF FIGURES

FIG. 1: shows the spectra of IR analysis of the model foam and the polyurea derived from PMDI (pMDI-urea) according to Example 1. On the abscissa, the wavenumber is shown in cm⁻¹ and on the ordinate the absorbance is given in arbitrary units.

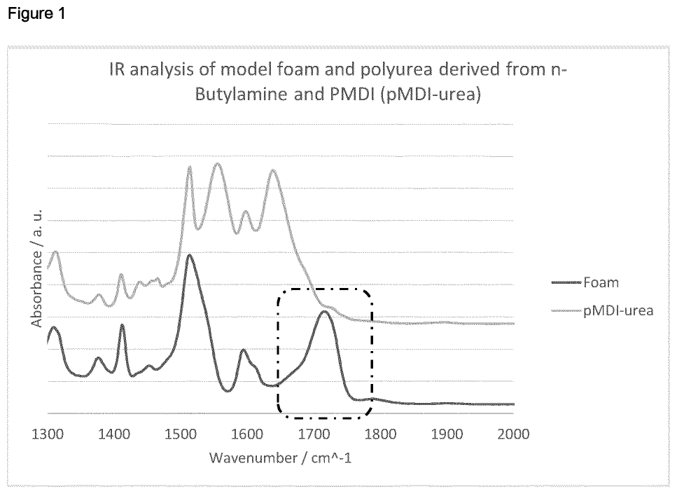

FIG. 2: shows the ¹³C-NMR spectrum of the polyurea derived from PMDI according to Example 1. On the abscissa, the chemical shift is given in ppm and on the ordinate the absorbance is given in arbitrary units.

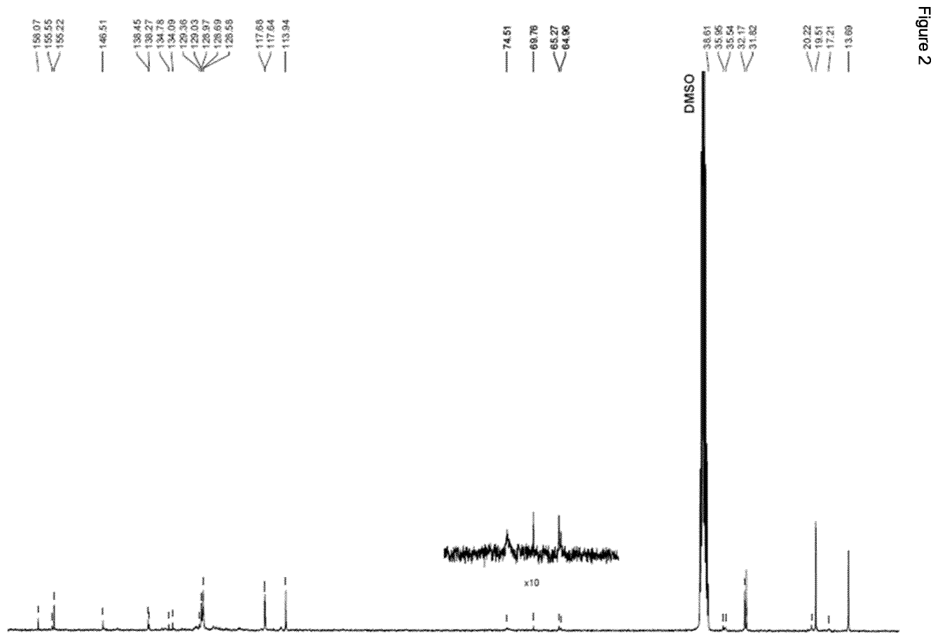

### CITED LITERATURE

WO 2008/014988 A1
“Kunststoffhandbuch [Plastics handbook], volume 7, Polyurethane [Polyurethanes]”, Carl Hanser Verlag, 3^{rd} edition 1993, chapter 3.2 and 3.3.2
WO 2006/034800
EP 0090444
WO 2005/090440

1.-17. (canceled)

18. A process for recycling one or more polymers selected from the group consisting of polyurethanes, polyurethane ureas, polyisocyanurates, and a mixture of two or more thereof, the one or more polymers being contained in a solid material W, the process comprising:
(i) providing the solid material W containing the one or more polymers;
(ii) introducing the solid material W and one or more primary amines in a reactor unit R_{A}, obtaining a mixture;
(iii) subjecting the mixture obtained according to (ii) in R_{A} to aminolysis reaction conditions, obtaining a mixture M comprising one or more polyurea-containing compounds, and further comprising one or more polyols;
(iv) isolating the one or more polyurea-containing compounds of M obtained according to (iii) from the one or more polyols of M obtained according to (iii), obtaining a mixture U comprising the one or more polyurea-containing compounds and a mixture P comprising the one or more polyols; and
(v) subjecting the mixture U comprising the one or more polyurea-containing compounds obtained according to (iv) to cleavage reaction conditions in a reactor unit R_{c}, obtaining either one or more corresponding polyamines or one or more corresponding polyisocyanates.

19. The process of claim 18, further comprising, prior to (i), subjecting the solid material W to a pre-treatment wherein the mechanical pre-treatment comprises one or more of milling, crushing, shredding and cutting the solid material W.

20. The process of claim 18, further comprising, prior to (i) drying the solid material W, wherein drying is conducted at a temperature in the range of from 40 to 100° C. in a gas atmosphere comprising one or more of nitrogen and oxygen.

21. The process of claim 18, wherein the one or more polymers contained in the solid material W are one or more polyurethanes.

22. The process of claim 18, wherein the one or more polymers contained in the solid material W are one or more polyisocyanurates or one or more polyurethane ureas.

23. The process of claim 18, wherein the reactor unit R_{A} according to (ii) comprises one or more reactors.

24. The process of claim 18, wherein the one or more primary amines are selected from the group consisting of aliphatic monoamines, aliphatic polyamines, aromatic monoamines, and mixtures of two or more thereof.

25. The process of claim 24, wherein the one or more primary amines are aliphatic monoamines having the formula H₂NR¹,
wherein R¹ is selected from the group consisting of (C₃-C₂₅) alkyl, phenyl, (C₇-C₂₅) aralkyl, and (C₇-C₂₅) alkaryl, and
wherein the aliphatic monoamines are selected from the group consisting of n-butylamines, cyclohexylamines, n-octylamines, n-hexylamines, n-propylamines, n-dodecylamines, ntridecylamines, n-octadecylamines, and mixtures of two or more thereof.

26. The process of claim 24, wherein the one or more primary amines are aromatic monoamines, wherein the aromatic monoamines are selected from the group consisting of aniline, toluidine, naphtylamine, and mixtures of two or more thereof.

27. The process of claim 18, wherein the ratio of the weight of the solid material W introduced into R_{A} relative to the weight of the one or more primary amines introduced into R_{A} is in the range of from 1:100 to 1:1.

28. The process of claim 18, wherein the aminolysis reaction according to (iii) is performed at a temperature in the range of from 50 to 250° C.

29. The process of claim 18, wherein the aminolysis reaction according to (iii) is performed at a pressure in the range of from 1.0 to 25.0 bar (abs).

30. The process of claim 18, further comprising, after (iii), removing the mixture M obtained according to (iii) from R_{A}; and further comprising prior to (v), passing the mixture M removed from R_{A} into a solid-liquid separation unit, obtaining a liquid mixture M_{SLS} comprising the one or more polyurea-containing compounds and the one or more polyols and a solid mixture comprising impurities.

31. The process of claim 18, wherein (iv) comprises:
(iv.1) passing a solvent and the mixture M into an evaporation unit EU, obtaining from EU a vapor mixture V comprising the solvent and at least a portion of the one or more primary amines, and a liquid mixture M_{EU} comprising the one or more polyurea-containing compounds and the one or more polyols;
(iv.2) adding a solvent to the mixture M, allowing the one or more polyurea-containing compounds to precipitate, obtaining a mixture comprising one or more precipitated polyurea-containing compounds; and
(iv.3) passing the mixture comprising the one or more precipitated polyurea-containing compounds obtained according to (iv.2) through a solid-liquid separation unit SLU, obtaining a liquid mixture P comprising the one or more polyols, and further obtaining a solid mixture U comprising the one or more urea-containing compounds.

32. The process of claim 31, wherein the solvent used in one or more of (iv.1) and (iv.2) is selected from the group consisting of hydrocarbons, ketones and ethers.

33. The process of claim 18, wherein the cleavage reaction conditions according to (v) are hydrolysis reaction conditions;
wherein (v) comprises:
admixing water with the mixture U comprising the one or more polyurea-containing compounds obtained according to (iv) in R_{c}, obtaining one or more corresponding polyamines.

34. The process of claim 18, wherein the cleavage reaction conditions according to (v) are acidic cleavage conditions;
wherein (v) comprises:
admixing a Bronsted acid, with the mixture U comprising the one or more polyurea-containing compounds obtained according to (iv) in R_{c}, obtaining one or more corresponding polyisocyanates.
